(19) United States
(12) Patent Application Publication
(10) Pub. No.: US 2018/0263829 A1
(43) Pub. Date: Sep. 20, 2018

(54) INCONTINENCE GARMENT ASSEMBLY

(71) Applicant: Earline Encalade, Darrow, LA (US)

(72) Inventor: Earline Encalade, Darrow, LA (US)

(21) Appl. No.: 15/459,415

(22) Filed: Mar. 15, 2017

(51) Int. Cl. A61F 13/62; A61F 13/49; A61F 13/53; A61F 13/491

(52) U.S. Cl. CPC A61F 13/622; A61F 13/4915; A61F 13/53; A61F 13/49011

(57) ABSTRACT

An incontinence garment assembly includes a band forming a closed loop. The band has an upper edge and a lower edge. A panel is attached to the bottom edge and extends downwardly therefrom. The panel has a distal edge and a pair of lateral edges. The panel is comprised of an absorbent material. The absorbent material includes a plurality of layers and the plurality of layers includes an outer layer is fluid impermeable. Each of a pair of straps has a first end attached to the band and a second end attached to the panel adjacent to the distal edge. The panel is positioned between the straps.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to incontinence garment devices and more particularly pertains to a new incontinence garment device for capturing urine released by a male suffering from urinary incontinence.

### BRIEF SUMMARY OF THE INVENTION

An embodiment of the disclosure meets the needs presented above by generally comprising a band forming a closed loop. The band has an upper edge and a lower edge. A panel is attached to the bottom edge and extends downwardly therefrom. The panel has a distal edge and a pair of lateral edges. The panel is comprised of an absorbent material. The absorbent material includes a plurality of layers and the plurality of layers includes an outer layer is fluid impermeable. Each of a pair of straps has a first end attached to the band and a second end attached to the panel adjacent to the distal edge. The panel is positioned between the straps.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front perspective view of a incontinence garment assembly according to an embodiment of the disclosure.

FIG. 2 is a rear view of an embodiment of the disclosure.

FIG. 3 is a cross-sectional view of an embodiment of the disclosure taken along line 3-3 of FIG. 2.

FIG. 4 is a front view of an embodiment of the disclosure.

FIG. 5 is a cross-sectional view of an embodiment of the disclosure taken along line 5-5 of FIG. 1.

### DETAILED DESCRIPTION OF THE INVENTION

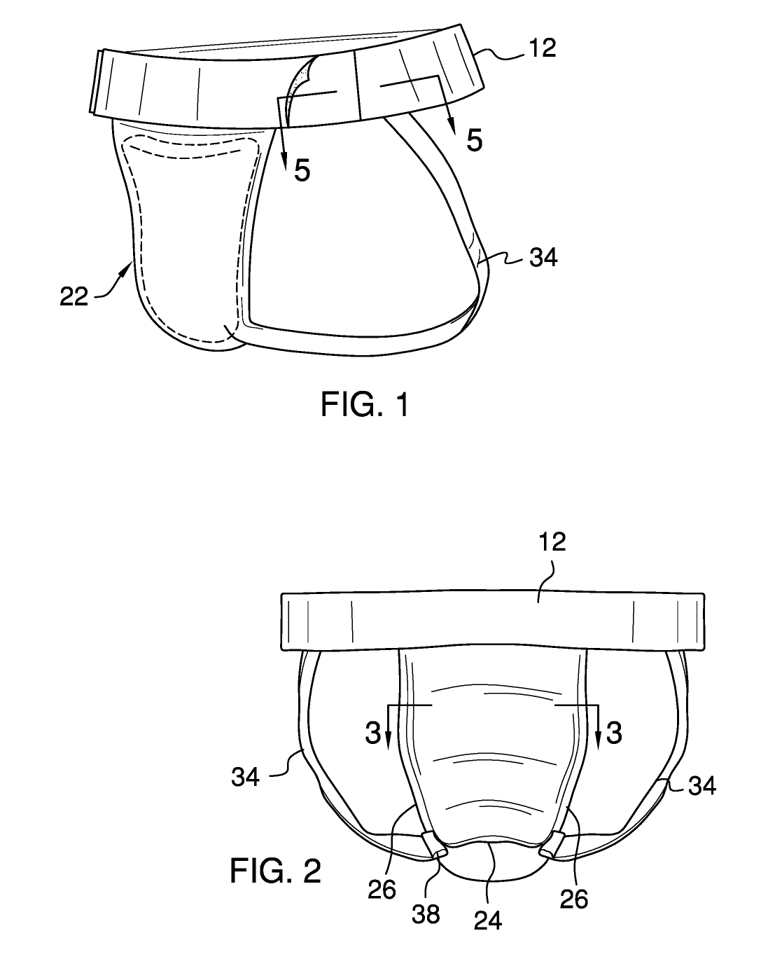

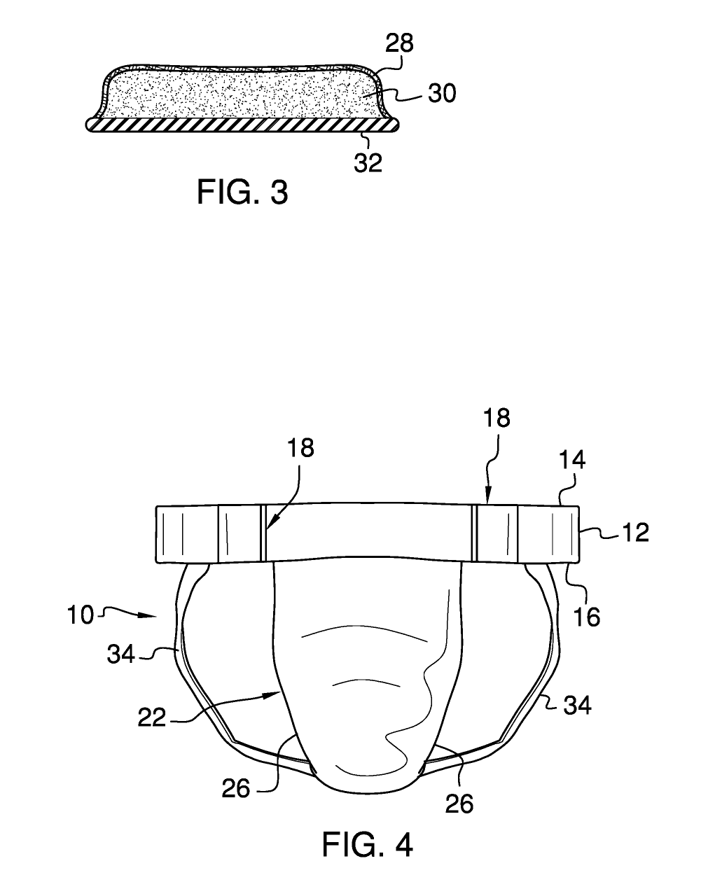

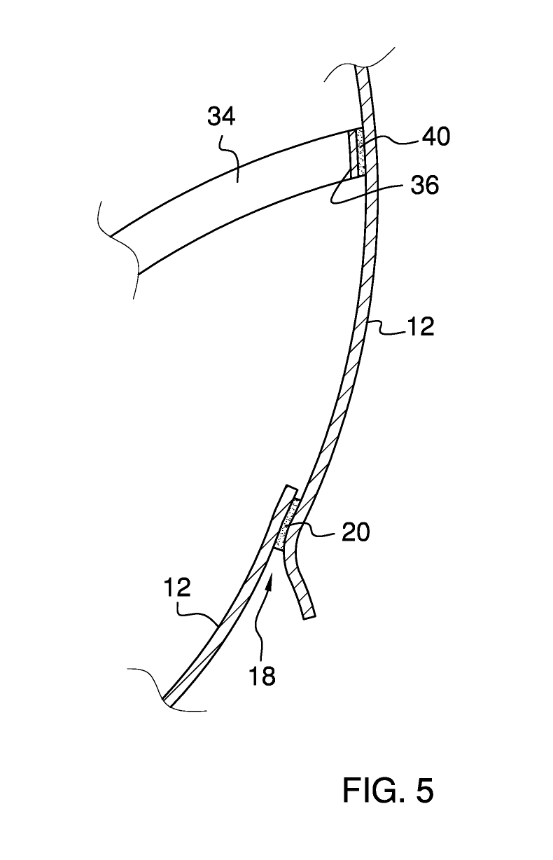

With reference now to the drawings, and in particular to FIGS. 1 through 5 thereof, a new incontinence garment device embodying the principles and concepts of an embodiment of the disclosure and generally designated by the reference numeral 10 will be described.

As best illustrated in FIGS. 1 through 5, the incontinence garment assembly 10 generally comprises a band 12 forming a closed loop. The band 12 has an upper edge 14 and a lower edge 16. The band 12 has a break 18 therein to facilitate opening the closed loop. A securing member 20 releasably retains the break 18 in a closed condition. The securing member 20 may comprise an adhesive, hook and loop coupler, or other conventional securing member. The band 12 may be comprised entirely or in part of a resiliently elastic material. As can be seen in the Figures, the break 18 may comprise a pair of breaks 18.

A panel 22 is attached to the band adjacent 12 to the bottom edge 16 and extends downwardly therefrom. The panel 22 has a distal edge 24 with respect to the band 20 and a pair of lateral edges 26. The panel 22 is comprised of an absorbent material. The absorbent material includes a plurality of layers 28, 30, 32. The plurality of layers includes an outer layer 32 that is fluid impermeable. The plurality of layers may further include a plurality of wicking materials 30 to draw moisture from an inner layer 28 toward the outer layer 32. The panel 22 has a width between the lateral edges 26 adjacent to the band 12 that is greater than 3.0 inches and less than 8.0 inches and the panel 22 has a height from the band 12 to the distal edge 24 that is greater than 5.0 inches and less than 10.0 inches. When worn, the panel 22 will preferably have a size and shape configured to cover the penis and extend below and under the testicles and thus may form a cup adjacent to the distal edge 24

A pair of straps 34 is provided each having a width less than 2.0 inches. Each of the straps has a first end 36 attached to the band 12 and a second end 38 attached to the panel 22 adjacent to the distal edge 24. The straps 34 may be comprised of a resiliently elastic material. The panel 22 is positioned between the straps 34. The first ends 36 are positioned between 4.0 inches and 12.0 inches from the panel 22. The first ends 36 may each be releasably attached to the band. This may be achieved with adhesive 40, snaps, hook and loop fasteners and the like.

In use, the assembly 10 is worn by persons suffering from urinary incontinence. The wearer extends their legs through the openings formed between the straps 34 and the panels 22 and uses the band 12 as a waistband. Should the panel 22 be soiled or the wearer simply wants to remove the assembly 1, the break 18 may be opened and the straps 34 separated from the band 12 so that the assembly 10 may be pulled from the wearer without the need of the wearer to remove their pants.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An incontinence garment assembly comprising:
a band forming a closed loop, said band having an upper edge and a lower edge;
a panel being attached to said bottom edge and extending downwardly therefrom, said panel having a distal edge and a pair of lateral edges, said panel being comprised of an absorbent material, said absorbent material including a plurality of layers, said plurality of layers including an outer layer being fluid impermeable; and
a pair of straps, each of said straps having a first end attached to said band and a second end attached to said panel adjacent to said distal edge, said panel being positioned between said straps.

2. The incontinence garment assembly according to claim 1, wherein said band has a break therein to facilitate opening said closed loop, a securing member releasably retaining said break in a closed condition.

3. The incontinence garment assembly according to claim 1, wherein said band is resiliently elastic.

4. The incontinence garment assembly according to claim 1, wherein said panel has a width between said lateral edges adjacent to said band being greater than 3.0 inches and less than 8.0 inches.

5. The incontinence garment assembly according to claim 4, wherein said panel has a height from said band to said distal edge being greater than 5.0 inches and less than 10.0 inches.

6. The incontinence garment assembly according to claim 1, wherein said panel has a height from said band to said distal edge being greater than 5.0 inches and less than 10.0 inches.

7. The incontinence garment assembly according to claim 1, wherein said first ends are each releasably attached to said band.

8. An incontinence garment assembly comprising:
a band forming a closed loop, said band having an upper edge and a lower edge, said band having a break therein to facilitate opening said closed loop, a securing member releasably retaining said break in a closed condition, said band being resiliently elastic;
a panel being attached to said bottom edge and extending downwardly therefrom, said panel having a distal edge and a pair of lateral edges, said panel being comprised of an absorbent material, said absorbent material including a plurality of layers, said plurality of layers including an outer layer being fluid impermeable, said panel having a width between said lateral edges adjacent to said band being greater than 3.0 inches and less than 8.0 inches, said panel having a height from said band to said distal edge being greater than 5.0 inches and less than 10.0 inches; and
a pair of straps, each of said straps having a first end attached to said band and a second end attached to said panel adjacent to said distal edge, said panel being positioned between said straps, said first ends each being releasably attached to said band.
